(19) United States
(12) Patent Application Publication
(10) Pub. No.: US 2007/0198035 A1
(43) Pub. Date: Aug. 23, 2007

(54) Method and surgical instrument operable for percutaneous ligation of a vein or vessel

(76) Inventor: Judson E. Threlkeld, Camas, WA (US)

Correspondence Address: KOLISCH HARTWELL, P.C., 200 PACIFIC BUILDING, 520 SW YAMHILL STREET, PORTLAND, OR, 97204, US

(21) Appl. No.: 11/356,966

(22) Filed: Feb. 17, 2006

(51) Int. Cl. A61B 17/04

(52) U.S. Cl. 606/148

(57) ABSTRACT

A surgical instrument and method for facilitating percutaneous ligation of a vein or vessel inside the body includes an elongate element having a bore extending longitudinally therethrough and a needle section mounted on the elongate element, and movable relative thereto, the needle section being provided with a channel for receiving a suture extending through the longitudinal bore so that an end of the suture is presented at an opening of the needle section. A retractor mounted adjacent the elongate element is operable for engaging the suture adjacent the opening of the needle section and for pulling the suture so that it engages a portion of the vein or vessel.

### BACKGROUND AND SUMMARY OF THE INVENTION

The present invention relates to a method and surgical instrument operable for providing percutaneous ligation of a vein or vessel, in surgical, endoscopic or laparoscopic procedures. The surgical instrument of the present invention is inserted in a minimally invasive percutaneous procedure, and this is accomplished by the novel construction of the surgical instrument.

### BRIEF DESCRIPTION OF THE DRAWINGS

FIG. 1 is a side, cross sectional view of the surgical instrument of the present invention after it has been percutaneously deployed, and prior to a needle portion of the instrument being rotated;

FIG. 2 is a view similar to FIG. 1, showing the needle end rotated into suitable position adjacent a vein so that an end of the suture may be captured by a retractor;

FIG. 3 shows deployment of the retractor for capturing an end of the suture;

FIG. 4 is a view similar to FIGS. 1 and 3, but shows the suture being fully retracted so that the captured end extends outwardly from the surgical instrument;

FIG. 5 shows extension of the needle end so that the percutaneously deployed surgical instrument may be withdrawn;

FIG. 6 shows the surgical instrument almost completely withdrawn; and

FIGS. 7-9 show the final steps of ligation of the vein.

### DETAILED DESCRIPTION OF THE PREFERRED EMBODIMENTS

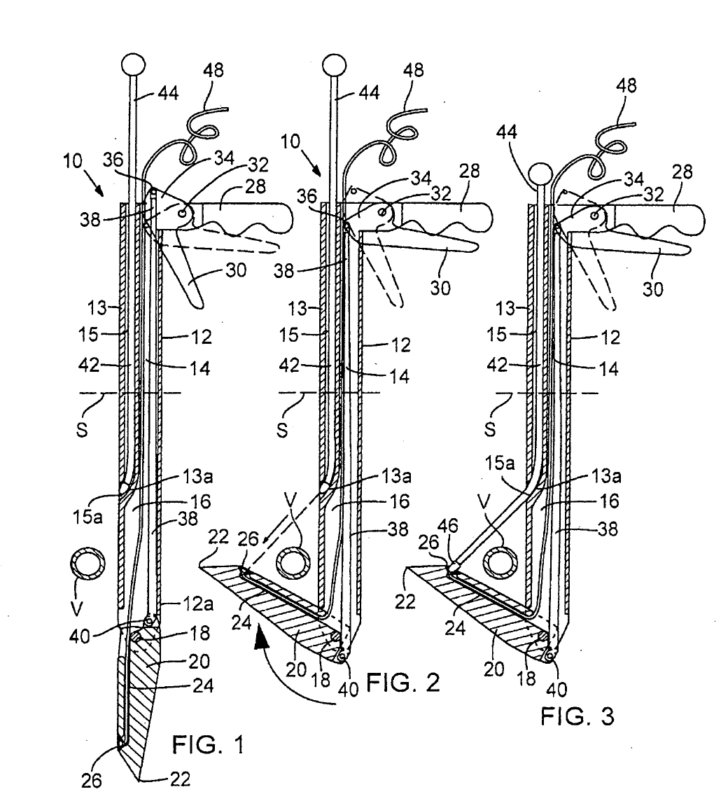

As shown in FIG. 1, a surgical instrument designed according to the present invention is generally designated at 10, and is shown inserted into a person's body adjacent a target vein or vessel to be sutured, indicated at V. The outer portion of a person's skin is shown at S, and before the method using the surgical instrument is explained in detail, features of the construction of the device will be described. The surgical instrument includes an elongate element such as a shaft 12, having a longitudinal bore 14 extending therethrough. As can be seen in FIG. 1, bore 14 transitions into a larger bore indicated at 16, adjacent a distal end 12a of the shaft. Pivotally mounted at 18 at the distal end of shaft 12 is a needle section 20 which has a beveled surface, ending in a sharpened point 22. Needle section 20 is shown as a rigid element provided with an elongate bore or channel 24 which communicates with bore 16 and also presents a side opening with a suture clasp at 26, for receiving a suture and engaging a retraction device in a manner to be described. Shown at the proximal end of shaft 12 is a handle 28 mounted thereto, which is provided with an actuator 30, pivotally connected to the handle at 32. The actuator includes a portion 34 which is pivotally connected at 36 to an elongate push rod 38 which extends downwardly through longitudinal bore 14 and enlarged bore section 16 for pivotal connection at 40 to an extension of needle section 20. The actuator is oriented to be manipulated by a surgeon, as will be described later, to extend or retract push rod 38 to thereby pivot needle section 20 about pivot connection 18.

FIG. 1 also shows that shaft 12 is provided with an auxiliary portion or shaft 13 which in turn may be formed as a tubular element having a longitudinal bore 15 extending therethrough. The purpose of bore 15 is to provide a region in which a retractor, such as an elongate rod 44, may be mounted for shifting therewithin. As shown, rod 44 is provided with an end 46, formed as a needle or similar construction, to engage suture clasp 26. Rod 44 is dimensioned for being pushed or directed outwardly from a lower opening of bore 15 indicated at 15a. End 46 is configured to capture a suture which is presented at suture clasp 26 of needle 24 at the appropriate sequence during percutaneous ligation of vein V. It will be noted that end 46 or rod 44 is received within opening 15a in a curved or bent configuration because bore 15 is curved so that opening 15a is diverted to face on the outside of shaft 13. The curved portion of bore 15 is defined by an inside wall of shaft 13 being curved as shown at 13a. As shown in FIG. 1, a suture is shown at 48, extending downwardly through bore 14, enlarged bore 16, further through channel 24 and then extended so that an end of the suture is presented at the suture clasp 26.

### Procedure Using the Surgical Instrument

The components of surgical instrument 10 were described with reference to FIG. 1, and as explained at the outset of the present application, the procedure contemplates the percutaneous ligation of a vein or vessel by inserting the needle into a position adjacent the vein, and actuating the needle in so that a suture presented at an opening of the needle is oriented for being engaged by a retractor. Retraction of the end of the suture outwardly through the skin then enables the ligation to be completed. The first step, with reference to FIG. 1, contemplates that a suture, such as indicated at 48, is suitably extended downwardly through bore 14, enlarged bore section 16 and through channel 24 so that an 3 end of a suture is presented at the opening at suture clasp 26 and held therein by a retainer. It is initially assumed that actuator 30 is oriented in a clockwise position so that rod 38 is urged upwardly so as to extend needle section 20 substantially in line with shaft 12, as illustrated. Retractor 44 is positioned so that end 46 is adjacent opening 15a, prior to insertion into the body.

It is to be understood that use of the surgical instrument will be facilitated by use of ultrasound guidance; in any case, a preselected vein or vessel, such as shown at V is targeted for ligation, and with surgical instrument oriented in the configuration shown in FIG. 1, the surgeon guides the beveled end preceded by sharpened point 22 downwardly through the skin, so that it is percutaneously deployed, as shown in FIG. 1. FIG. 2 shows that actuator 30 has been manipulated by the surgeon so that needle section 20 is pivoted clockwise in the position shown to rotate beneath vein V. While FIG. 1 shows that the needle section is substantially aligned with shaft 12, as the needle section descends below vein V, it should be appreciated that a surgeon could simultaneously initiate pivoting of needle section 20 as the surgical instrument is inserted, with a goal being to arrive in the position shown in FIG. 2.

In the position illustrated by FIG. 2, suture clasp 26, which is connected to suture 48, is presented so that it is in line, as indicated by the dashed line in FIG. 2 at 52, so that deployment downwardly of retractor 44 will position end 46 of the retractor so that it will engage and capture the end of suture 44. The full deployment of retractor 44 is shown in FIG. 3, whereby capture has been completed. It will be noted in FIG. 3 that rod 44 has been guided to the proper position, and this may be ensured by the curved end portion of bore 15, or retractor 44 may be designed with a prestressed configuration so that the required angle to effectuate capture is provided.

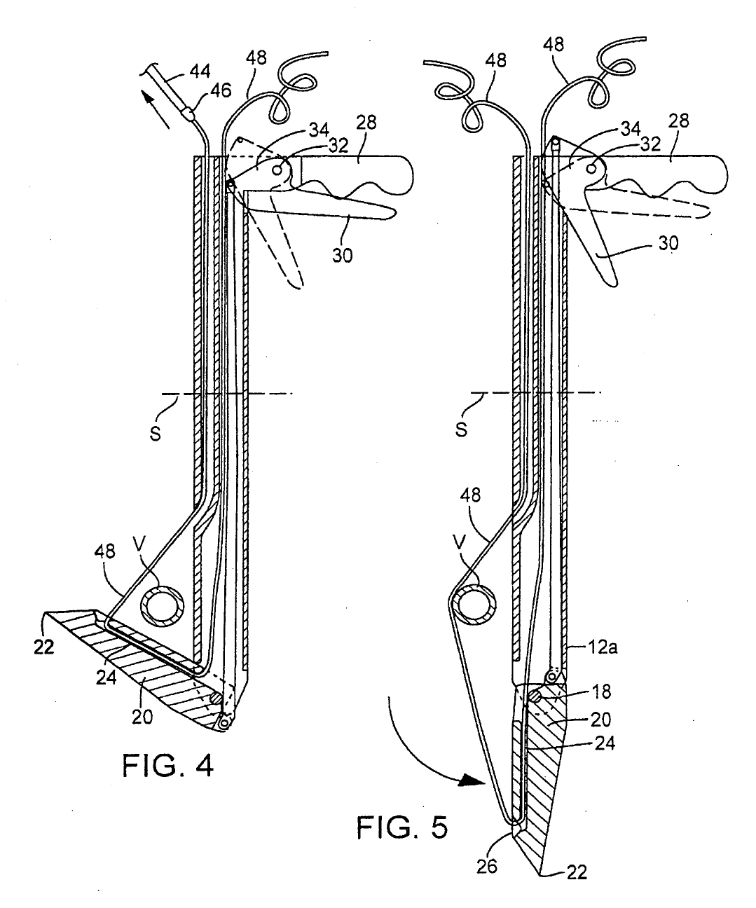

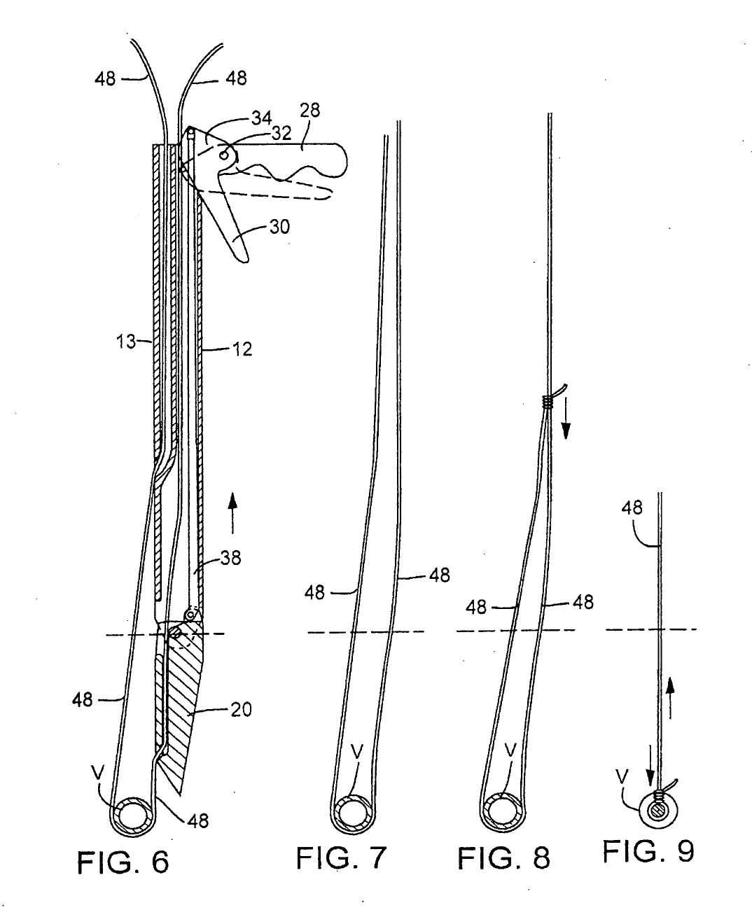

As shown in FIG. 4, retractor 44 is retracted so that suture 48 is pulled upwardly through bore 15 as shown, and under proper guidance by the surgeon, the surgical instrument is suitably operated so that needle section 20 is extended, as shown in FIG. 5, and the device may be withdrawn, as shown in FIG. 6, now with the suture wrapping around a portion of vein V. The surgical instrument is removed, and through appropriate procedures, the surgeon forces a slipknot to ligate the vein as shown in the steps of FIGS. 8 and 9. Slipknots and the procedures conforming to their use for ligation are old in the art; it is the surgical instrument and the specific method steps of the percutaneous ligation which are believed novel in the present invention.

Thus, in recap, the procedure utilizing the present invention contemplates the following steps, utilizing the tool as herein above described. Initially, the surgical instrument is operably deployed by the surgeon so that an end of a suture, held by a clasp device at an opening in the needle section, is moved to a position on an opposite side of the vein whereby a retractor, having a needle end can be directed toward the clasp device for retracting or withdrawing the suture. When the suture is withdrawn, the needle of the surgical instrument may be realigned, the surgical device removed from adjacent the vein, outwardly from the body and a slipknot utilized for ligation of the vein upon suitable tensioning of the suture.

I claim:

1. A surgical instrument for facilitating percutaneous ligation of a vein or vessel inside the body comprising:
an elongate element having a bore extending longitudinally therethrough;
a needle section mounted on the elongate element, and movable relative thereto, the needle section provided with a channel for receiving a suture extending through the longitudinal bore, so that an end of the suture is presented at an opening of the needle section;
a retractor mounted adjacent the elongate element operable for engaging the suture adjacent the opening of the needle section and pulling the suture so that it engages a portion of the vein or vessel.

2. The surgical system of claim 1 wherein an actuator is provided on the elongate element operable to move the needle section from a position substantially aligned with the elongate member to a nonaligned position so that the end of the suture maintained at the opening of the needle section is positionable on one side of the vein or vessel.

3. The surgical instrument of claim 2 wherein the needle section is pivotally connected adjacent an end of the elongate member so that when the elongate member is inserted into the body, adjacent a vein or vessel, the needle section may be pivoted by the actuator to present the end of the suture maintained at the opening of the needle section on one side of the vein or vessel.

4. The surgical instrument of claim 3 wherein the retractor is elongate, and may be manipulated by a surgeon for selected positioning within a bore positioned adjacent the elongate element.

5. The surgical instrument of claim 4 wherein the actuator includes a rod which is extendable/retractable within the longitudinal bore of the elongate member, and which is connected to the needle section.

6. A method for the percutaneous ligation of a vein or vessel comprising the steps of:
guiding the end of a suture through an elongate element and presenting an end thereof in an opening in a needle section mounted at the end of the elongate element adjacent a vein or vessel;
moving the needle section so that the end of the suture adjacent the opening in the needle section is on an opposite side of the vein or vessel;
engaging the end of the suture at the end of the opening in the needle section and pulling it so it engages a portion of the vein or vessel.
